(19) United States
(12) Patent Application Publication
(10) Pub. No.: US 2012/0035558 A1
(43) Pub. Date: Feb. 9, 2012

(54) Wound Cleaning Adhesive Patch

(76) Inventor: Peter R. POPA, (US)

(21) Appl. No.: 13/198,576

(22) Filed: Aug. 4, 2011

Related U.S. Application Data
(60) Provisional application No. 61/371,476, filed on Aug. 6, 2010.

(51) Int. Cl. A61L 15/44; A61M 35/00

(52) U.S. Cl. 604/290; 604/367

(57) ABSTRACT

A patch that is able to expedite the healing process of skin wounds such as cuts, burns, scraps, or any other superficial wounds. The present invention is an adhesive patch with gauze retaining a suds substance. The patch is applied to a user's skin wound to clean and draw out the pus and exudates for a quicker healing process.

The current application claims a priority to the U.S. Provisional Patent application Ser. No. 61/371,476 filed on Aug. 6, 2010.

### FIELD OF THE INVENTION

The present invention relates generally to a wound patch. More specifically, a skin wound patch for accelerated healing.

### BACKGROUND OF THE INVENTION

During the healing process of a regular skin wound, burn, open cut, etc. exudate, environmental contaminates, pus, etc., accumulate over the wound as a response to the injury. In most cases, people immediately apply antibiotics, dry sponge coverings, creams, oil based substances, etc. to the wound without removing any of the wound's waste material. Even after cleaning the wound with soap, water and other substances micro-contamination of the wound persists and continuously transudate, exudate and possibly pus keep forming during the healing process. Because pus and/or exudates including bacteria, cellular debris, proteic components of inflammatory response, environmental particles, etc. slow the healing of the wound, draining of such components would remove a lot of potential harmful material sitting in the affected area. Therefore, removal of such is necessary in order to expedite the healing. The present invention uses gauze or sponge-like material soaked in soap- or detergent-like suds to speed the healing process by removing the pus and waste. The surrounding adhesive film will stick on the healthy skin and will isolate the wound from the environment thereby preventing the suds from drying out and keeping the sponge or gauze in contact with the wound surface.

### BRIEF DESCRIPTION OF THE DRAWINGS

FIG. 1 is a perspective view of the present invention in a circular embodiment.

FIG. 2 is a perspective view of the present invention in a square embodiment.

FIG. 3 is a view of the present invention applied to a skin wound. The triangular particles are the exudates and pus from the skin wound. The circular particles are the suds substances. The suds substances are shown dissolving and drawing the exudates away from the skin wound.

### DETAIL DESCRIPTIONS OF THE INVENTION

All illustrations of the drawings are for the purpose of describing selected versions of the present invention and are not intended to limit the scope of the present invention.

The present invention is a wound patch that is mostly intended but not restricted for superficial skin wounds such as abrasions, burns, etc. The present invention is especially useful for skin wounds with or without signs of infection. However, the wound patch is also used for the prevention of infection and wound cleaning where the skin barrier was broken and an inflammatory repairing process takes place.

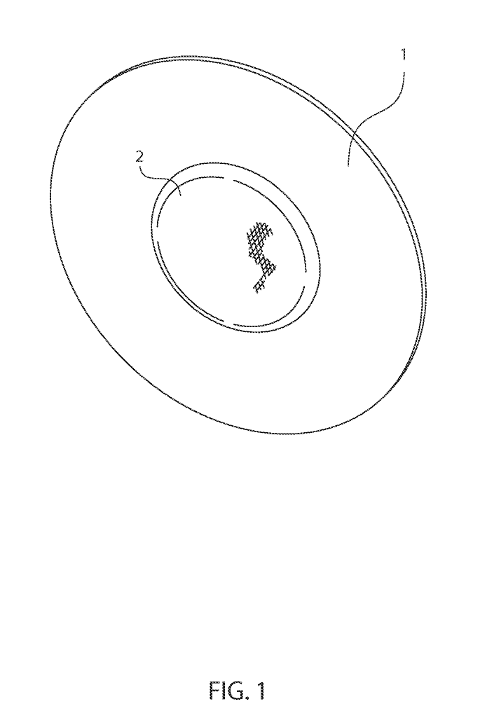

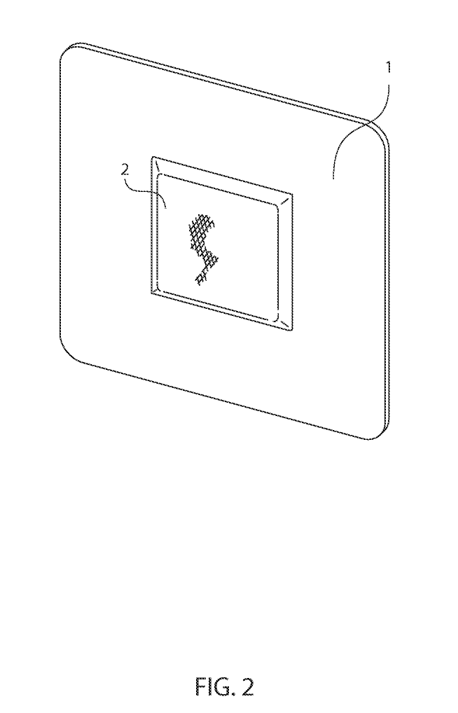

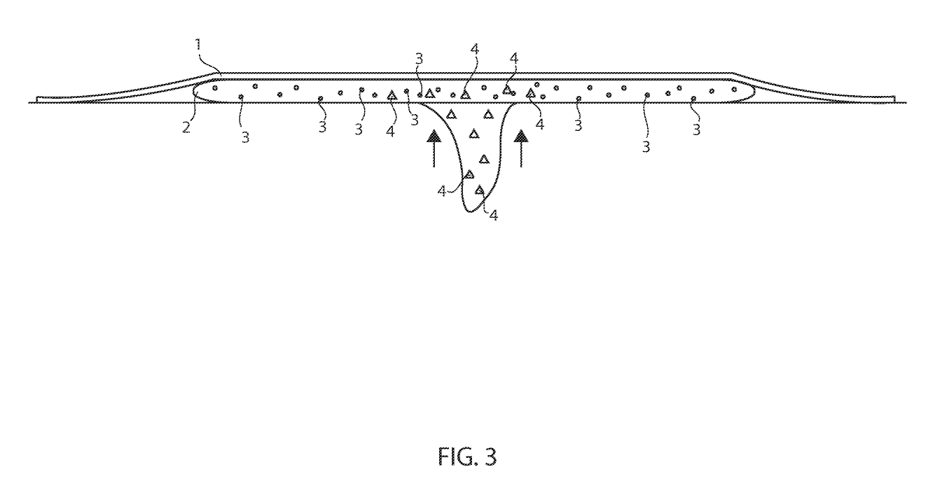

In reference to FIG. 1-3, the present invention is a wound cleaning adhesive patch that comprises an adhesive film 1, a soaked pad 2, with a suds substance 3. The adhesive film 1 having an adhesive side and a non-adhesive side is used for the protection and sealing of the user's skin wound. The soaked pad 2 is adhered to and positioned on the adhesive side of the adhesive patch. The soaked pad 2 can be any absorbing material including gauze, sponge-like materials, or any other suitable absorbing material for application to wounds. The soaked pad 2 is centered on the adhesive film 1 to ensure that the wound is completely sealed by the adhesive film 1. The suds substance 3 can be any soap or detergent like solution which dissolves the above mentioned waste material and contaminates, solution that may or may not be antibacterial or contain antibiotic substances. The size and shape of the present invention can vary to accommodate the size and shapes of different wounds. The suds substance 3 is retained by the soaked pad 2.

To use the present invention on a skin wound, it is important to perform a cleansing of the wound to remove any obvious external contaminants and dead tissue from the wound. The soaked pad 2 is then aligned and placed over the wound. The adhesive film 1 is then adhered to the user's skin around the skin wound. As a result the wound and the soaked pad 2 are sealed and prevent any additional bacteria, foreign substances, or other environmental factors from infecting and contaminating the wound. The sealing of the skin wound and the soaked pad 2 by the adhesive film 1 also prevents the drying of the suds substance 3. The pus, exudates 4, etc., and other foreign substances, are drawn in by the suds substance 3 and the soaked pad 2 by dissolving and diffusing it from a higher to lower concentration. As the wound excretes pus and/or exudates, the soaked pad 2 is able to physically dissolve lipophilic material and remove it by diffusion, absorbing any dirt, cellular debris, proteic components from inflammatory responses, environmental particles, etc. from the surface of the wound. The suds substance 3 acts as a solvent facilitating the absorbing process through diffusion of the dissolved material from a higher to a lower concentration towards the pad. As a result, the pus and exudates 4 are drawn away from the skin wound, leaving the wound clean for an expedited healing process.

The principle of the present invention is the application of the suds substance 3 onto a wound by means of the soaked pad 2. The soapsuds are applied on the wound for a period of time under an occlusive dressing. This process will moisten the wound's fibrin crust. and remove the pus, exudates 4, and other foreign substance from the wound by dissolving it into the suds that are in contact with the surface of the wound. Even in the absence of any antibacterial properties of the applied soap/detergent suds, the application of the soap/detergent like material will have a beneficial healing effect by cleaning effectively the wound and helping the healing process.

The present invention has been tested on several patients with exceptional results. In one embodiment of the present invention, sterile gauze is scrubbed with a regular hypoallergenic soap to the desired scrub consistency. The patch is then applied to the wound and covered with the adhesive film 1. The adhesive film 1 can be a polymer film that is flexible and can easily conform to the user's skin. The adhesive film 1 is waterproof and moisture vapor impermeable to prevent outside contamination and drying of the soaked pad 2. After 24 hours, upon the removal of the wound cleaning adhesive patch, the wound is left clean off all of the pus and exudates 4 which are dissolved into the suds substance 3 in the soaked pad 2. The process can be repeated several times until the wound is considered clean. The present invention is extremely valuable also in providing symptom relief by decreasing pain and signs of inflammation due to removal of inflammatory proteins and thereof diminishing the nociceptive effect to provide symptom relief at the wound site.

The difference between the present invention and traditional wound dressings on the market is that the present invention applies a dressing containing soap/detergents like suds. The suds substances 3 contained in the soaked pad 2 are able to drain and cleanse the wound. In comparison, wound dressings including ones that use antibiotics, dry sponge coverings, creams, alcohol or oil based substances do not facilitate pus, exudates , etc. 4 away from a skin wound.

In many cases, the infected open wounds require prescription of systemic antibiotics. The use of the present invention decreases the need for the use of systemic therapy. Additionally, with reduced usage of systemic antibiotics, the chances of bacterial resistance of developing are also decreased. Reduced antibiotic usage also decreases the money a patient must spend on antibiotics. However, in other cases, the present invention can also be used in conjunction with systemic antibiotic treatment by ameliorating symptoms, facilitating healing and decreasing healing time. The present invention is also most efficient with use on wound with a larger superficial area, the present invention is still applicable to superficial small wounds or puncture wounds.

In another application of the present invention, the wound cleaning adhesive patch can be used for injuries such as burn wounds where the skin barrier has been compromised. Even if signs of infection are not visible, the removal of the exudates will deplete the bacteria from an excellent growth medium and in conjunction with the wound sealing will significantly decrease the chances of infection. Burn wounds are also superficial and can be prone to infection due to large amount of exudates/transudates 4.

Although the invention has been explained in relation to its preferred embodiment, it is to be understood that many other possible modifications and variations can be made without departing from the spirit and scope of the invention as hereinafter claimed.

What is claimed is:

1. A wound cleaning adhesive patch comprises, an adhesive film;
a soaked pad;
a suds substance; the soaked pad being an absorbent material selected from the group consisting of gauze, dressing, or-sponges;
the soaked pad being adhered to and positioned on the adhesive film;
the suds substance being retained by the soaked pad; and
the soaked pad being centered on the adhesive film.

2. A method of using the wound cleaning adhesive patch for a sterile and expedited healing process comprises,
providing an wound cleaning adhesive patch, wherein the wound cleaning adhesive patch comprises a adhesive film and a soaked pad;
wherein the soaked pad retains a suds substance which preferably but not necessarily has antibacterial properties;
placing the soaked pad over a skin wound;
adhering the adhesive film around the skin wound;
sealing of the skin wound with the soaked pad by the adhesive film to prevent drying of the suds substance.

3. The method of using the wound cleaning adhesive patch for a sterile and expedited healing process as claimed in claim 2 comprises,
wherein the adhesive film protects the wound from the environment.

4. The method of using the wound cleaning adhesive patch for a sterile and expedited healing process as claimed in claim 2 comprises,
wherein the skin wound excretes transudate, exudate, pus, etc.; and
wherein the excreted transudate, exudates, pus, etc. is dissolved by the suds substance and subsequently the dissolved lipophilic material is drawn away by diffusion from the wound.

5. A method of using the wound cleaning adhesive patch for a sterile and expedited healing process comprises,
providing a wound cleaning adhesive patch, wherein the wound cleaning adhesive patch comprises a adhesive film and a soaked pad;
wherein the soaked pad retains a suds substance;
placing the soaked pad over a skin wound;
adhering the adhesive film around the skin wound;
sealing of the skin wound with the soaked pad by the adhesive film to prevent drying of the suds substance;
wherein the adhesive film protects the wound from the environment;
wherein the skin wound excretes pus and/or exudates;
wherein the excreted pus and exudates is dissolved by the antibacterial suds substance and drawn away from the skin wound.
